(19) United States
(12) Patent Application Publication
(10) Pub. No.: US 2006/0140888 A1
(43) Pub. Date: Jun. 29, 2006

(54) Whitening agent containing crystalline molecular complex of hydroquinone and surfactant

(76) Inventors: Yuji Ohashi, Tokyo (JP); Nahoko Iimura, Niigata-shi (JP)

Correspondence Address: Millen White Zelano & Branigan;Arlington Courthouse Plaza 1, Suite 1400, 2200 Clarendon Blvd, Arlington, VA, 22201, US

(21) Appl. No.: 10/527,078

(22) PCT Filed: Sep. 10, 2003
(86) PCT No.: PCT/JP03/11590

§ 371 Date: Nov. 22, 2005

(30) Foreign Application Priority Data
Sep. 10, 2002 (JP) 2002-26436

(51) Int. Cl. A61K 8/41; A61K 8/34

(52) U.S. Cl. 424/62

(57) ABSTRACT

There are provided a hydroquinone-containing whitening agent having high storage stability and sustained release of hydroquinone, a process for production of the whitening agent, and a whitening method for skin which employs the whitening agent. The whitening agent of the invention comprises a crystalline molecular complex composed of hydroquinone or a derivative thereof and a surfactant, wherein formation of the molecular complex improves the storage stability of the hydroquinone-containing whitening agent against heat, oxygen and light, while the hydroquinone is gradually released for a sustained whitening effect of the whitening agent.

### FIELD OF THE INVENTION

The present invention relates to a whitening agent containing a crystalline molecular complex comprising hydroquinone or a derivative thereof and a surfactant, characterized in that formation of the molecular complex improves the storage stability of the hydroquinone-containing whitening agent against heat, oxygen and light, while the hydroquinone is gradually released during use for a sustained whitening effect of the whitening agent, as well as to a process for production of the whitening agent, to the use of the surfactant for production of the whitening agent, and to a skin whitening method whereby the whitening agent is applied to pigmented skin.

### BACKGROUND ART

It is generally understood that skin blemishes, bruises and sunburn occur by a mechanism in which melanin pigment is formed as a result of hormonal imbalance or stimulation by ultraviolet sunlight, upon which the formed melanin pigment is abnormally deposited in the skin. One method for treating blemishes and bruises has been to administer or apply substances which inhibit melanin production, such as vitamin C, glutathione and cysteine. Yet these substances have very minimal whitening effects.

Hydroquinone and its derivatives are generally accepted as exhibiting whitening effects, unlike the substances mentioned above. However, hydroquinone and its derivatives also readily undergo coloration by air oxidation and the like and their inclusion in cosmetic materials has therefore been associated with various problems.

Hydroquinone (1,4-benzenediol; 1,4-dihydroxybenzene) is a white crystalline chemical substance having the structure shown in FIG. 1 (CA[123-31-9], Kashin No. 3-543, C₆H₆O₂=110.11, melting point: 170-171° C., boiling point: 285-287° C., d 1.332). It is readily soluble in methanol and ether, soluble in water and poorly soluble in benzene and ethyl acetate, and is gradually colored by air oxidation, producing quinhydrone.

Hydroquinone products found in Europe and the U.S. contain 2-4% hydroquinone as a whitening component for creams produced from ordinary cosmetic materials, which are marketed as “hydroquinone creams”. Hydroquinone creams are restricted in their use, for example, being indicated for only nighttime application or for daytime use only in combination with a sun blocking cream. This is presumably because the susceptibility of hydroquinone to the effects of oxygen and light is a problem that has not been satisfactorily overcome. One method that has been employed to avoid oxidation of hydroquinone involves sealing it with nitrogen prior to shipment and storing it in a sealed, light-protected container, but once the container is opened it is impossible to avoid exposure to oxygen and light during subsequent storage. Addition of antioxidants and the like has been shown to help prevent this, but according to certain reports, such addition can sometimes lead to skin roughening.

According to Oojima et al. in “Treatment of hypermelanosis with hydroquinone external applications” (Nishinihon J. of Dermatology, Vol. 42, No. 1, 1980), virtually all the hydroquinone compounds included in drug components marketed by cosmetic manufacturers for a certain time after World War II were monobenzyl ether hydroquinones (MEHQ), but upon successive reports of treatment side effects with long-term use of hydroquinone, such as leukoderma and depigmentation resembling vitiligo vulgaris, formulation of hydroquinone compounds in cosmetics was later prohibited in 1957 by Pharmaceutical Affairs Law No. 534. Hydroquinone formulations have therefore not been marketed as products in Japan, although they are sold by Elder Co. in the U.S. under the names Eldoquin and Eldopaque, which are listed as containing 2% hydroquinone. oojima et al. reported that HQ external applications turn completely yellowish-brown in about two weeks when stored in an ointment can at ordinary temperature, but that stability is greatly improved by addition of 3% L-ascorbic acid to prevent oxidation and immediate transfer to a tube, and further that upon use promptly after storage in a butter chamber in the door of a refrigerator, no degeneration-associated side effects were experienced by treatment application during the previous 3 years.

Patricia G. et al. teach, in “Cosmetics and dermatology: Bleaching creams” J. Am. Acad. Dermatol. 5:143-147(1981), that hydroquinone-containing whitening creams are effective and stable at concentrations of 2-5%, but that users are given careful directions with regard to its use and protection from sunlight. Also, combined use with superficial local corticosteroids, salicylic acid or tretinoin by physician prescription has been reported to significantly improve the whitening effects of hydroquinone-containing whitening creams.

In “Investigating hydroquinone ointments”, Iyaku Journal Vol. 20, No. 10, pp. 1929-1934(1984), Ueda et al. teach that 2% hydroquinone ointments (HQ ointments) are used as demelanizing treatment for chloasma, freckles, Riehl's melanosis and pigmentation following exanthema, but that HQ readily undergoes auto-oxidation and blackish-brown discoloration, constituting an inconvenience for its use. Ueda et al. also reported that addition of both citric acid and sodium sulfite as antioxidants, or addition of acidic sodium sulfite alone, successfully prevented discoloration to allow prolonged storage.

In “Hydroquinone ointment quality and clinical evaluation”, JJSHP, Vol. 24, No. 7,8(1998), Karashima et al. explains that since a HQ formulation (Wako Junyaku Special Reagents) is readily auto-oxidized by light and air and becomes discolored to brown, sodium bisulfite, vitamin C (VC) and the like are used as preventive antioxidants, but that skin allergies have been reported with the use of HQ ointments with VC added. Karashima et al. therefore prepared and pharmaceutically evaluated HQ ointments using different bases and HQ ointments comprising VC. It was reported that an HQ ointment employing plastibase (PL, Taisho Pharmaceutical) as the base was stable without temperature effects, but other bases such as official hydrophilic ointment (HP) and official absorbent ointment (Ab) underwent coloration with time, and HQ ointments using HP and D-1-0 (decaglycerin monooleate gel) as bases were resistant to coloration when containing VC and stored at 4° C.

In “(2) Burn treatment ointments and pigmentation treatment ointments”, Gekkan Yakuji, Vol. 38, No. 12 (1996), Matsuhara et al. report that hydroquinone ointments are used for hypermelanosis at most facilities, and that the bleaching effect of hydroquinone monobenzyl ether is so strong as to cause leukoderma, such that currently only hydroquinone is used clinically (see non-patent document 5). It is further stated that application of hydroquinone ointments must be undertaken with care because of the side effect of sunlight-sensitive pigmentation increase.

In “Structures of complex crystals of alkylammonium salts with aromatic molecules”, Mol. Cryst. Lig. Cryst., 1996, Vol. 276, pp. 185-191, Noguchi K. et al. describe the results of X-ray diffraction analysis of molecular complex crystals composed of dodecyltrimethylammonium chloride (LTAC) and catechol, and those composed of LTAC and hydroquinone. However, absolutely no mention is made regarding the stability of hydroquinone.

In “Treatment of post inflammatory pigmentation using retinoic acid”, Keisei Geka 42(4): 297-301, 1999, Yoshimura et al. report on the use of hydroquinone external applications for treatment of post inflammatory pigmentation. A 5% hydroquinone/7% lactic acid plastibase formulation was unstable and was therefore prepared once a month and stored in a cold, dark place, and the hydroquinone/lactic acid ointment was applied to the affected area of patients twice a day, together with a sun block cream during the daytime; care had to be taken to avoid urtication and dermatitis with particularly high concentrations of the external hydroquinone.

In “Research on whitening agents”, Fragrance Journal 2001-3 pp. 65-66 (translation), Zhai H. et al. explain that hydroquinone is available in the U.S. as an OTC (over-the-counter) drug up to a 2.0% concentration and as a prescription drug at greater concentrations, and that hydroquinone-containing creams are efficacious.

In “Evaluating usefulness of pigmentation treatment ointments”, Iyaku Journal, Vol. 37, No. 2, pp. 807-812(2001), Tanaka et al. report that hydroquinone-based ointments exhibiting melanin production-inhibiting effects had been prepared for several years at different institutions for pigmentation such as senile pigment macules and nevus spilus and had been used in the clinic, and discusses the pharmaceutical evaluation and therapeutic effects of the preparations for outpatients received at dermatology clinics. A high proportion of patients using hydroquinone ointments judged them to be effective, while the incidence of side effects was surprisingly low. In addition, combination of chemical peeling or ruby laser treatment with the ointments produced satisfactory effects for the aforementioned symptoms, although commuting to the hospital for treatment was an inconvenience in some cases and the desire was often expressed for an easier treatment using the ointment alone; the conclusion was therefore that a need exists for formulation of a pigmentation treatment ointment as a more powerful treatment drug with low side effects, to allow rapid curing of various hypermelanosis conditions.

In “New combined treatment of hypermelanosis: Analytical studies on efficacy and stability improvement”, International Journal Cosmetic Science (2002) 23/6 (333-340), V. Ferioli et al. examine the whitening effects of hydroquinone and kojic acid. In order to increase the stability of the hydroquinone in the ointment, a complex is formed with β-cyclodextrin, and this complex is stable against heat as determined by DSC, HSM and X-ray diffraction. However, β-cyclodextrin, a donut-shaped molecule which forms clathrates by enclosing compounds in its molecule, is not a general surfactant.

Japanese Unexamined Patent Publication (Kokai) SHO No. 61-271204 discloses a liposome formulation comprising a hydroquinone glucoside without the oxidation-coloring disadvantage of hydroquinone, embedded in a lamellar phase of liposomes comprising natural and synthetic phospholipids and negatively and positively charged complex lipids (including chemical or physical adsorption onto the lamellar phase surface). This publication mentions stability of the hydroquinone glucoside as a whitening agent and selective migration and sustained release onto affected areas. It also teaches that yellowing due to oxidation of the hydroquinone skeleton is prevented by the liposomes.

Japanese National Patent Publication (Kohyo) No. 2001-520652 discloses a composition comprising a sustained release molecular complex composed of an α-hydroxy acid and an organic chelating agent, and its use as an external application. It also describes the whitening effects of hydroquinone monomethyl and benzyl ester derivatives as added active substances. An aromatic compound is included as the α-hydroxy acid. A non-amphoteric amino acid amide or the like is suggested as the organic chelating agent, and in the case of alkyl substitution at either or both of the hydrogen atoms of the amino acid amide, aliphatic amides defined as surfactants are included within the generic concept. However, this molecular complex is a molecular complex of an α-hydroxy acid and an organic chelating agent, and not a crystalline molecular complex of hydroquinone and a surfactant.

Also, Japanese Patent Application No. 2000-118551, a prior application by the present inventors, discloses a method of forming molecular complex crystals composed of a surfactant and different aromatic compounds, to inhibit the volatilization rate of the aromatic compounds. Although hydroquinone is included among the aromatic compounds used, the disclosure is not particularly directed toward hydroquinone and it contains absolutely no suggestion or description for improving the stability against oxidation and light.

Thus, hydroquinone is known as an effective whitening agent, and hydroquinone creams are widely used in Europe and the U.S. In Japan, however, hydroquinone monobenzyl ether and hydroquinone have been considered to be identical components, and therefore hydroquinone has been withheld from marketing as an extremely dangerous chemical substance. Yet in recent years dermatologists have come to employ it in the clinic as a powerful blemish treatment, and as its sundry effects have begun to be corroborated, the whitening effect of hydroquinone is gradually gaining acceptance. For effective product development, however, it is essential to solve the problems of reduced storage stability and skin irritation of hydroquinone-containing formulations and products, caused by oxidation and light. If a solution could be found for these problems, then, hydroquinone sustained release whitening products with high storage stability could be developed.

### DISCLOSURE OF THE INVENTION

The present inventors have completed this invention as the result of diligent research aimed at solving the problems described above, to determine whether a hydroquinone sustained release whitening product with high storage stability can be produced by forming a crystalline molecular complex of hydroquinone and a surfactant.

In Japanese Patent Application No. 2000-118551 referred to above, the present inventors discovered that crystalline molecular complexes are formed between surfactants and various aromatic compounds, and we have elucidated their crystalline structures. Also, we discovered that the aromatic compounds which have formed crystalline molecular complexes with the surfactants are more resistant to volatilization caused by increasing temperature, even in very high temperature ranges, than are the aromatic compounds alone and that appropriate selection of the type of surfactant can optimally inhibit the volatilization rate (sustained release).

In addition to this discovery, the present inventors have now confirmed that aromatic compounds which have formed crystalline molecular complexes with surfactants are even more protected from oxidation and light than are the aromatic compounds alone, and the present invention has been completed by applying the aforementioned discovery to hydroquinone as the active ingredient in a whitening agent, for confirmation of the effect.

According to a first aspect of the invention, there is provided a whitening agent comprising a crystalline molecular complex composed of hydroquinone or a derivative thereof and a surfactant, characterized in that formation of the molecular complex improves the storage stability of the hydroquinone-containing whitening agent against heat, oxygen and light, while the hydroquinone is gradually released for a sustained whitening effect of the whitening agent.

The aforementioned hydroquinone or its derivative may be selected from the group consisting of hydroquinone, hydroquinone monobenzyl ether, hydroquinone monomethyl ether and hydroquinone monoethyl ether.

The aforementioned hydroquinone or its derivative is preferably hydroquinone.

The surfactant may be selected from the group consisting of octadecyltrimethylammonium bromide (STAB), octadecyltrimethylammonium chloride (STAC), hexadecyltrimethylammonium bromide (CTAB), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC) (also known as benzylacetyldimethylammonium chloride), tetradecyldimethylbenzylammonium bromide (BZB), tetradecyldimethylbenzylammonium chloride (BZCL), dodecyltrimethylammonium bromide (LTAB), dodecyltrimethylammonium chloride (LTAC), decyltrimethylammonium bromide (DTAB), decyltrimethylammonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

The surfactant is preferably selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

More preferably, the surfactant is CDBAC.

According to a second aspect of the invention, there is provided the use of a crystalline molecular complex composed of hydroquinone or a derivative thereof and a surfactant for production of a whitening agent, characterized in that formation of the molecular complex improves the storage stability of the hydroquinone-containing whitening agent against heat, oxygen and light, while the hydroquinone is gradually released for a sustained whitening effect of the whitening agent.

The aforementioned hydroquinone or its derivative may be selected from the group consisting of hydroquinone, hydroquinone monobenzyl ether, hydroquinone monomethyl ether and hydroquinone monoethyl ether.

The aforementioned hydroquinone or its derivative is preferably hydroquinone.

The surfactant may be selected from the group consisting of octadecyltrimethylammonium bromide (STAB), octadecyltrimethylammonium chloride (STAC), hexadecyltrimethylammonium bromide (CTAB), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC), tetradecyldimethylbenzylammonium bromide (BZB), tetradecyldimethylbenzylammonium chloride (BZCL), dodecyltrimethylammonium bromide (LTAB), dodecyltrimethylammonium chloride (LTAC), decyltrimethylammonium bromide (DTAB), decyltrimethylammonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

The surfactant is preferably selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

More preferably, the surfactant is CDBAC.

According to a third aspect of the invention, there is provided a skin whitening method wherein a whitening agent comprising a crystalline molecular complex composed of hydroquinone or a derivative thereof and a surfactant is applied to pigmented skin, the method being characterized in that formation of the molecular complex improves the storage stability of the hydroquinone-containing whitening agent against heat, oxygen and light, while the hydroquinone is gradually released for a sustained whitening effect of the whitening agent.

The aforementioned hydroquinone or its derivative may be selected from the group consisting of hydroquinone, hydroquinone monobenzyl ether, hydroquinone monomethyl ether and hydroquinone monoethyl ether.

The aforementioned hydroquinone or its derivative is preferably hydroquinone.

The surfactant may be selected from the group consisting of octadecyltrimethylammonium bromide (STAB), octadecyltrimethylammonium chloride (STAC), hexadecyltrimethylammonium bromide (CTAS), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC), tetradecyldimethylbenzylammonium bromide (BZB), tetradecyldimethylbenzylammonium chloride (BZCL), dodecyltrimethylammonium bromide (LTAB), dodecyltrimethylammonium chloride (LTAC), decyltrimethylammonium bromide (DTAB), decyltrimethylammonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

The surfactant is preferably selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

More preferably, the surfactant is CDBAC.

According to a fourth aspect of the invention, there is provided a process for production of the aforementioned whitening agent, which comprises the following steps:

dispersing a crystalline molecular complex composed of hydroquinone or a derivative thereof and a surfactant in a first oil phase;

preparing a second oil phase;

preparing an aqueous phase;

adding the aqueous phase to the second oil phase and stirring to form an emulsion; and

adding the first oil phase containing the molecular complex to the emulsion and stirring to obtain a whitening cream containing the aforementioned molecular complex.

The first oil phase may contain mineral oil, white vaseline, liquid paraffin, polyoxyethylene (2) stearyl ether and/or polyoxyethylene stearyl ether stearate.

The second oil phase may contain mineral oil, jojoba oil, glycol distearate, polyoxyethylene (25) stearyl ether, polyoxyethylene isostearyl ether, sorbitan tristearate, octamethylcyclotetrasiloxane, tristearin, stearic acid, squalane and/or cetanol.

The aqueous phase may contain glycerin, 1,3-butanediol, trehalose, citric acid and/or EDTA-2Na, and purified water.

### BRIEF DESCRIPTION OF THE DRAWINGS

FIG. 1 is structural diagram showing hydroquinone and a surfactant used for preparation of molecular complex crystals.

FIG. 2 is an illustration of the molecular structure of a CDBAC/hydroquinone molecular complex.

FIG. 3 is a crystal structure diagram (a axis projection) of a CDBAC/hydroquinone molecular complex.

FIG. 4 is a crystal structure diagram (b axis projection) of a CDBAC/hydroquinone molecular complex.

FIG. 5 is a graph showing oxidation of simple hydroquinone and the aforementioned molecular complex crystals, at 37° C.

FIG. 6 is a graph showing thermal stability of simple hydroquinone and surfactant/hydroquinone molecular complex crystals.

FIG. 7 is a graph showing effects of light at 25° C. on the CDBAC/HQ, BZCl/HQ and CTAC/HQ molecular complex crystals, and simple HQ, prepared in Example 1.

FIG. 8 is a set of photographs in lieu of a drawing, showing the outer appearance of simple ointments containing 3% of different surfactant/HQ molecular complex crystals, after standing for 3 months in air at room temperature.

FIG. 9 is a set of photographs in lieu of a drawing, showing the outer appearance of hydrophilic ointments containing 1-2% of different surfactants/HQ molecular complex crystals, after standing for 2 weeks in air at room temperature.

FIG. 10 is a photograph in lieu of a drawing, showing the results after a 48-hour patch test (Nos. 1-10).

FIG. 11 is a photograph in lieu of a drawing, showing the results after a 48-hour patch test (Nos. 11-15).

FIG. 12 is a photograph in lieu of a drawing, showing the results after a 72-hour patch test (Nos. 1-10).

FIG. 13 is a photograph in lieu of a drawing, showing the results after a 72-hour patch test (Nos. 11-15).

FIG. 14 is a photograph in lieu of a drawing, showing the outer appearances of creams produced according to a conventional method or Example 6, at the time of preparation.

FIG. 15 is a photograph in lieu of a drawing, showing the outer appearances of creams produced according to a conventional method or Example 6, after standing for 24 hours at 40° C.

FIG. 16 is a photograph in lieu of a drawing, showing the outer appearances of creams produced according to a conventional method or Example 6, after standing for 72 hours at 40° C.

FIG. 17 a photograph in lieu of a drawing, showing the outer appearances of creams produced according to a conventional method or Example 6, after standing for 110 hours at 40° C.

FIG. 18 is a graph showing the results of measuring (at 40° C.) the a* value for cream coloration using a differential calorimeter.

FIG. 19 is a graph showing the results of measuring (at 40° C.) the L* value for cream coloration using a differential calorimeter.

FIG. 20 a graph showing the results of measuring (at 40° C.) the b* value, with cream coloration using a differential calorimeter.

FIG. 21 is a photograph in lieu of a drawing, showing the results of a light resistance test with complex-containing cream prepared by the preparation method described in Example 6.

FIG. 22 is a photograph in lieu of a drawing, showing the results of a light resistance test with complex-containing cream prepared by a conventional preparation method.

FIG. 23 is a photograph in lieu of a drawing, showing the results of a light resistance test with a simple hydroquinone cream prepared by a conventional preparation method.

FIG. 24 is a table showing the results of measuring coloration of a light resistance test sample in Example 9 using a differential calorimeter.

### BEST MODE FOR CARRYING OUT THE INVENTION

Various surfactants such as ionic surfactants and anionic surfactants may be used with hydroquinone in appropriate molar ratios, and solubilized by an ordinary solubilizing method or both dissolved in an appropriate organic solvent and allowed to stand at an appropriate temperature, to obtain a molecular complex of the surfactant and hydroquinone in crystal form. The crystalline molecular complex obtained in this manner is more stable against heat, oxygen and light than is simple hydroquinone, and by using a surfactant with a long alkyl chain length it is possible to suppress the release rate of hydroquinone from the molecular complex. This permits control of the sustained release property of hydroquinone.

The present invention drastically provides improvement over the disadvantageous properties of hydroquinone, a whitening component whose efficacy for blemish treatment has been confirmed and is supported through the world. Specifically, the invention achieves enhanced storage stability of whitening agents and improved sustained release properties for the whitening component. As a result, the daily dosage of such whitening agents employed by users may be reduced, and user concerns about the side effects of hydroquinone can be alleviated. According to the invention, therefore, consumers can safely use the agent without following special directions as indicated in the past, and it becomes possible to develop hydroquinone-containing whitening products such as those which can be easily purchased in drugstores throughout Europe and the U.S.

Molecular complex crystals composed of hydroquinone and a surfactant (octadecyltrimethylammonium chloride (STAC), hexadecyltrimethylammonium bromide (CTAB), hexadecyltrimethylammonium chloride (CTAC), tetradecyltrimethylammonium chloride (MTAC), hexadecyldimethylbenzylammonium chloride (CDBAC), tetradecyldimethylbenzylammonium bromide (BZB), tetradecyldimethylbenzylammonium chloride (BZCL) or n-dodecyl-β-D-maltoside (DM)) can be prepared in the following manner.

Hydroquinone is added in an equimolar amount to a surfactant aqueous solution or alcohol solution under a nitrogen stream to produce a uniform solution, which is then allowed to stand for 3-7 days in a cool area at 2-3° C., and the resulting precipitate is isolated to obtain surfactant/hydroquinone molecular complex crystals.

The obtained molecular complex crystals are adequately dried and dissolved in methanol, the absorbance is measured at a specific absorption wavelength using a UV-visible spectrophotometer (UV160A, Shimadzu), and the value is compared with the absorbance for simple hydroquinone to confirm formation of the crystalline molecular complex.

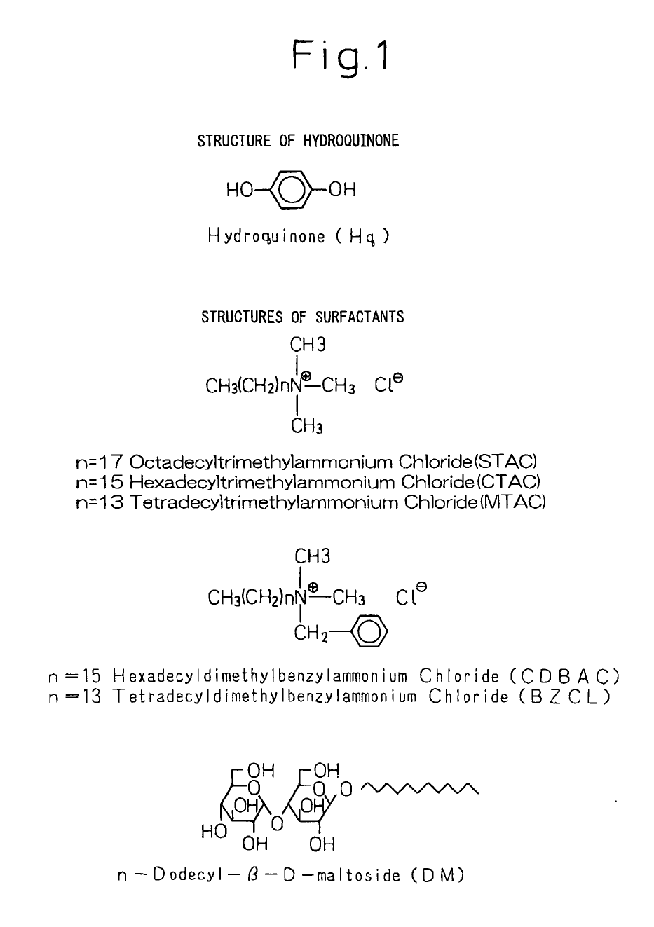

FIG. 1 shows the structure of hydroquinone and the structures of surfactants which can be used for preparation of molecular complex crystals. The surfactants used for the invention are not limited to those shown in FIG. 1. Hexadecyldimethylbenzylammonium chloride (CDBAC) is also known as benzylacetyldimethylammonium chloride, and it is a widely accepted surfactant listed in the Japan Comprehensive Licensing Standards Of Cosmetics by Category.

The production of molecular complex crystals can be confirmed by X-ray crystallographic analysis of the CDSAC/HQ crystals. The crystals are cooled to −50° C. using a nitrogen-blown cooling apparatus, and then analyzed with an imaging plate single crystal automatic X-ray structural analyzer (RAPID, Rigaku), using monochromated MoKa radiation. The phase is determined using the program SIR-97 by the direct method, and refined with the least-squares method using SHELXL-97 program. As an example, the crystallographic data for CDBAC/hydroquinone (HQ) molecular complex are shown in Table 1 below.

TABLE 1
Crystallographic data for CDBAC/hydroquinone
molecular complex

| Chemical formula | C₁₉H₄₂NCl/1.5C₆H₈O₂ | |
|---|---|---|
| Molecular weight | 506.19 | |
| a/Å | 18.3719 | (5) |
| b/Å | 7.0309 | (2) |
| c/Å | 50.6482 | (13) |
| β/° | 91.1170 | (10) |
| V/Å³ | 6541.0 | (3) |
| Space group | C2/c | |
| Z | 8 | |
| R | 0.0725 | |

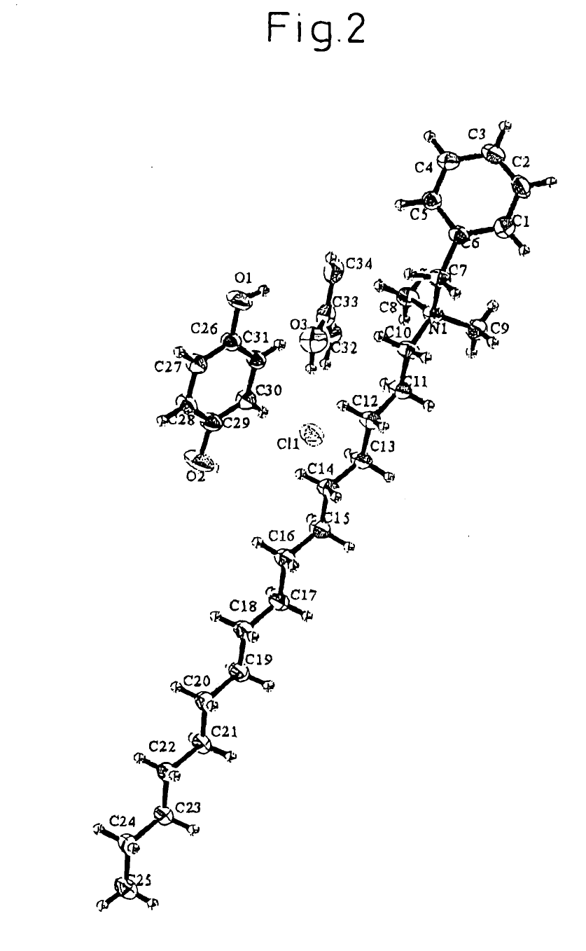

An illustration of the molecular structure of the CDBAC/hydroquinone molecular complex is shown in FIG. 2.

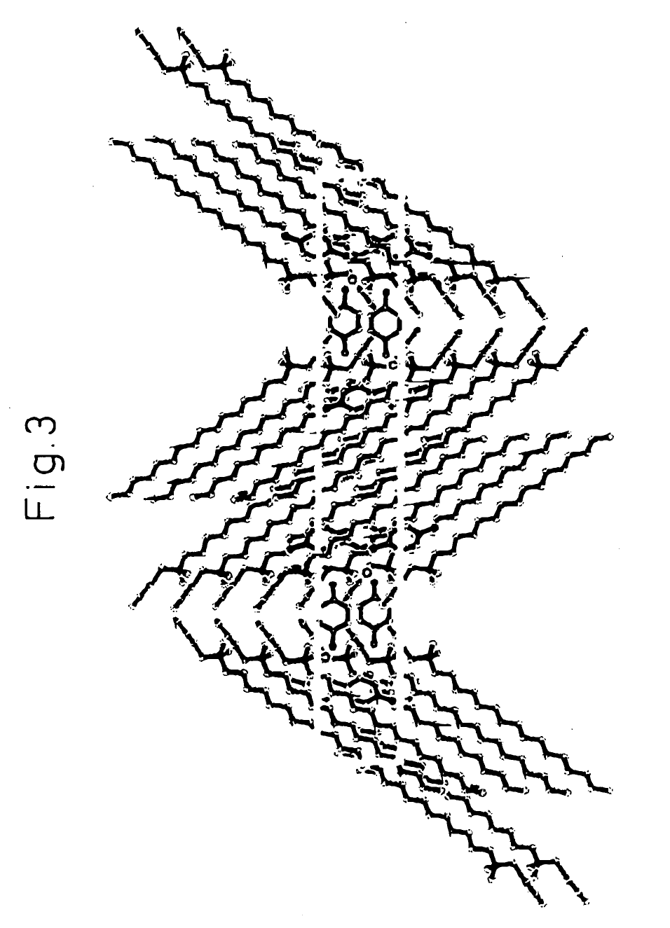

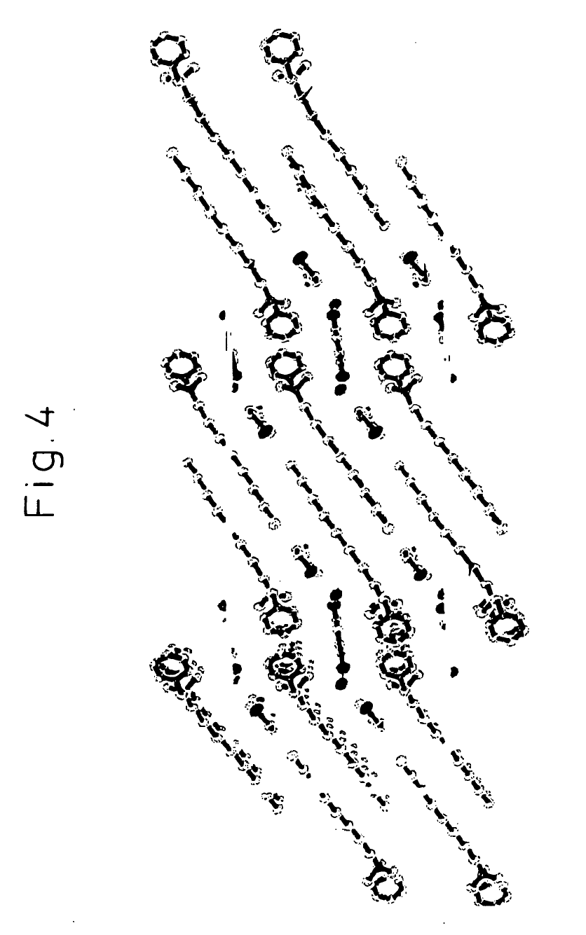

A crystal structure diagram of the CDBAC/hydroquinone molecular complex (a axis projection) is shown in FIG. 3, and the b axis projection is shown in FIG. 4.

From the crystallographic data shown above and the accompanying crystal structure diagram, it can be confirmed that the obtained crystals had formed the aforementioned molecular complex.

For preparation of a whitening agent according to the invention, it is necessary to prepare a whitening formulation which does not deteriorate in quality during long periods, retaining the hydroquinone/surfactant molecular complex style (crystal structure).

Most products marketed as whitening agents have been external applications for skin. Such whitening creams, beauty solutions and cosmetic water products are widely used as cosmetic products and quasi drugs.

Production of a cream, in cases where the whitening component (base component) is water-soluble, generally requires first dissolving or dispersing the base component in an oil phase and then either adding an oil phase to the obtained aqueous phase or an aqueous phase to the obtained oil phase and using a surfactant or the like for emulsification of the two immiscible phases for dispersion.

The hydroquinone/surfactant molecular complex used for the invention has an unstable crystal structure when water is present, and therefore measures must be taken so that the crystal structure of the molecular complex is not destroyed during such a production process in which water is present.

Specifically, it is believed that the hydroquinone/surfactant molecular complex in aqueous solution forms micelles to avoid repulsion between the surfactant alkyl chains and water. The micelles are dynamic, and it is assumed that the hydroquinone molecules can move freely in the micelles. Thus, since it is impossible to completely maintain the hydroquinone/surfactant style (crystal structure) in aqueous solution, the property of simple hydroquinone is exhibited. When hydroquinone is present by itself in a whitening agent, the whitening agent will of course be highly susceptible to product deterioration, including coloration. Consequently, for production of an external application for skin requiring addition of water (aqueous phase), this method by which the hydroquinone/surfactant molecular complex is added is extremely significant.

First, the hydroquinone/surfactant molecular complex is homogenized using a portion of the oil phase component. This can prevent dispersion of the simple hydroquinone from the molecular complex. Next, an oil S phase containing the molecular complex is added to the previously formulated and prepared base emulsion, and homogenized. In order for the whitening agent obtained in this manner to maintain a weakly acidic pH to prevent coloration, it is also important to actively suppress structural changes in the hydroquinone.

### EXAMPLES

### Example 1

### Comparison of Effects of Oxygen on Molecular Complex Crystals Composed of Hydroquinone and Surfactant (octadecyltrimethylammonium chloride (STAC), hexadecyltrimethylammonium chloride (CTAC) tetradecyltrimethylammonium chloride (MTAC), hexadecyldimethylbenzylarnmonium chloride (CDBAC), or tetradecyldimethylbenzylammonium chloride (BZCL)) and simple hydroquinone

Simple hydroquinone and different surfactant/hydroquinone molecular complex crystals were sorted to a particle size of 48-80 mesh and allowed to stand in a thermostatic bath at 37° C. with periodic sampling, and after dissolution in methanol, the absorbance was measured at a specific absorption wavelength using a UV-visible spectrophotometer (UV160A, Shimadzu), and deterioration of hydroquinone from the starting point was confirmed.

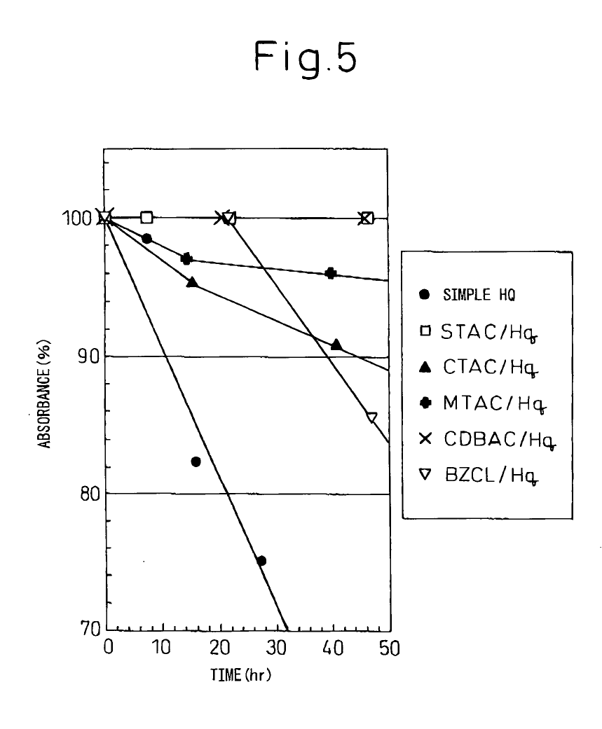

FIG. 5 is a graph showing oxidation of simple hydroquinone and the aforementioned molecular complex crystals, at a set temperature of 37° C. for approximation of human body temperature.

When compared with simple hydroquinone, the molecular complexes formed with the surfactants clearly suppressed oxidation of hydroquinone.

### Example 2

### Measurement of Thermal Stability of Molecular Complex Crystals Composed of Surfactant (hexadecyldimethylbenzylammonium chloride (CDBAC)) and hydroquinone

A Rigaku TG8120 (apparatus and manufacturer name) was used to measure the reduction in the number of moles of hydroquinone in the molecular complex crystals with increasing temperature, under a nitrogen stream in a temperature range of 25-160° C. at a temperature elevating rate of 10 K/min.

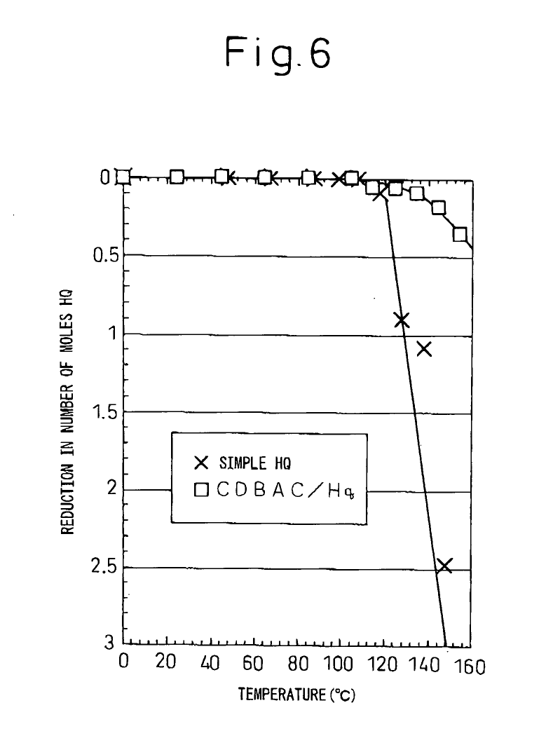

FIG. 6 is a graph showing thermal stability of simple hydroquinone and surfactant/hydroquinone molecular complex crystals. As seen in FIG. 6, volatilization of hydroquinone with increasing temperature was clearly suppressed by formation of the molecular complex crystals with the surfactant.

As explained in Japanese Patent Application No. 2000-118551, the suppression of volatilization is proportional to the length of the alkyl chain of the surfactant used, and this is theoretically supported by calculation results of van der Waals energy of molecular complex crystals based on Lennard-Jones potential. Thus, by appropriate selection of the type of surfactant it is possible to suppress the volatilization rate, or in other words, to control the sustained release of hydroquinone.

### Example 3

### Confirmation of Effect of Light on Surfactant/Hydroquinone Molecular Complex Crystals at 25° C.

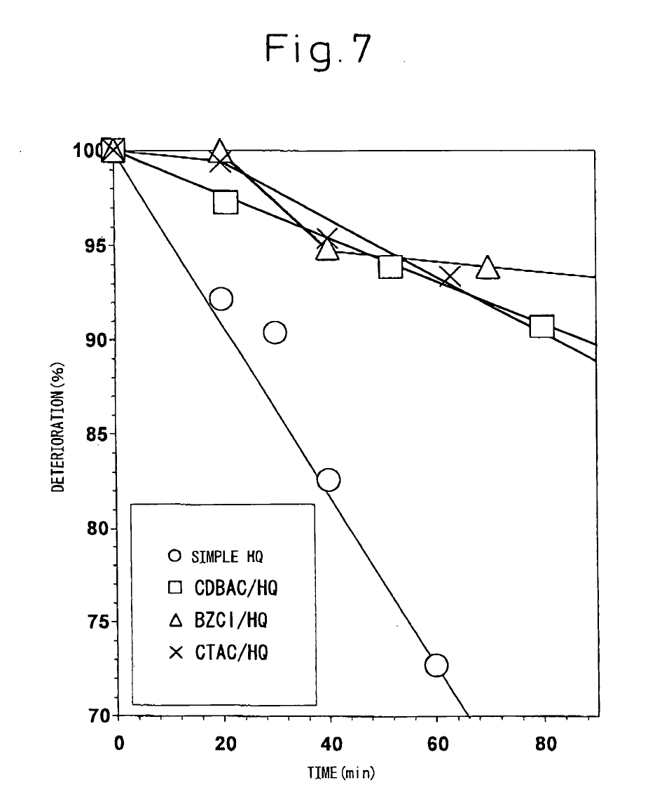

FIG. 7 is a graph showing effects of light at 25° C. on CDBAC/HQ, BZCl/HQ and CTAC/HQ molecular complex crystals, and on simple HQ.

Simple hydroquinone and different surfactant/hydroquinone molecular complex crystals were sorted to a particle size of 48-80 mesh, and then 0.01 g was weighed into a polyethylene bag and the bag was sealed using a Vacuum Sealer (VS-400, As-ONE) after sufficient deairing. A xenon lamp, Super Bright 152S (SAN-ELECTRIC), was then used for light irradiation at 30 mW/cm². After periodic sampling and dissolution in methanol, the absorbance was measured at a specific absorption wavelength using a UV-visible spectrophotometer (UV160A, Shimadzu), and deterioration of hydroquinone from the starting point was confirmed.

### Example 4

### Storage Sstability (Outer Appearance Change) of Ointments Prepared by Combining Different Surfactant/Hydroquinone Molecular Complex Crystals with Ointment Bases (Simple Ointment, Hydrophilic Ointment)

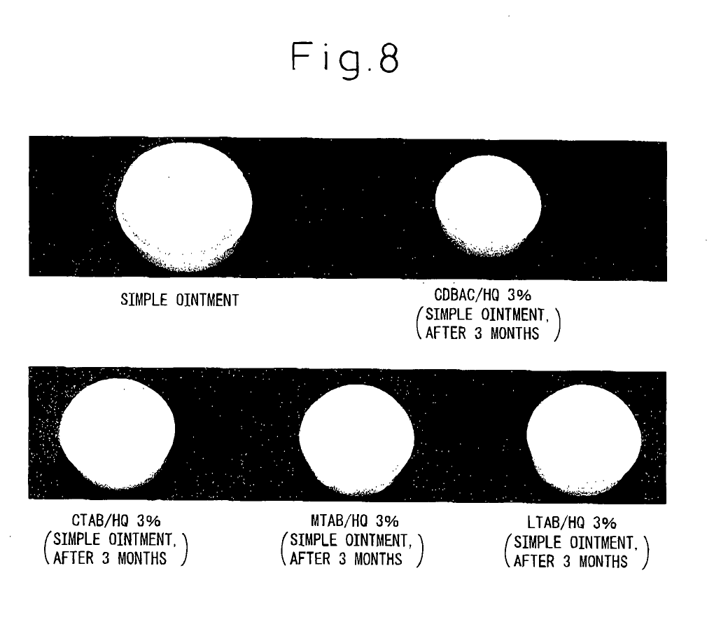

FIG. 8 shows the outer appearances of a simple ointment (upper left), a simple ointment containing 3% hexadecyldimethylbenzylammonium chloride (CDBAC)/HQ molecular complex crystals (upper right), a simple ointment containing 3% hexadecyltrimethylammonium bromide (CTAB)/HQ molecular complex crystals (lower left), a simple ointment containing 3% tetradecyltrimethylammonium bromide (MTAB)/HQ molecular complex crystals (lower center) and a simple ointment containing 3% dodecyltrimethylammonium bromide (LTAB)/HQ molecular complex crystals (lower right), after standing for 3 months in air at room temperature. All of the molecular complex crystal-containing simple ointments exhibited the same color hue as the simple ointment alone, clearly indicating inhibition of coloration by hydroquinone.

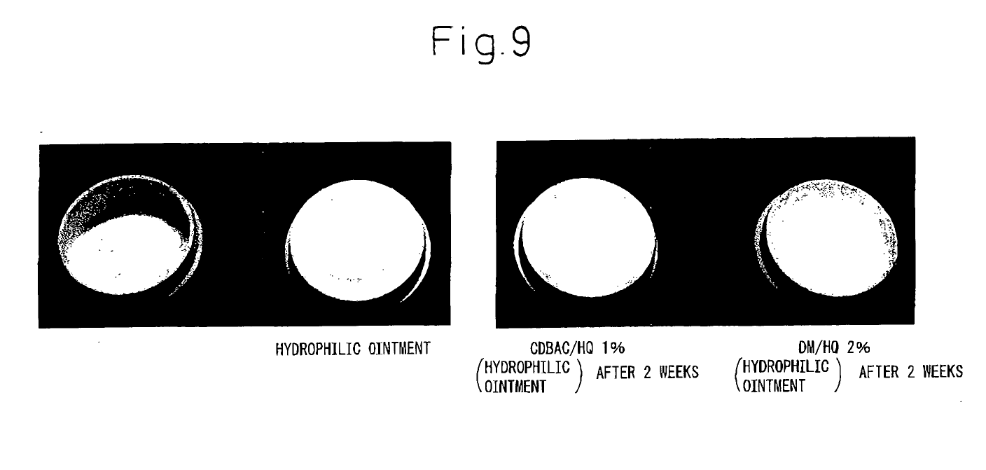

FIG. 9 shows the outer appearances of a hydrophilic ointment containing 5% simple hydroquinone (far left), a hydrophilic ointment alone (second from left), a hydrophilic ointment containing 1% hexadecyldimethylbenzylammonium chloride (CDBAC)/HQ molecular complex crystals (third from left) and a hydrophilic ointment containing 2% n-dodecyl-β-D-maltoside (DM)/HQ molecular complex crystals (far right), after standing for 2 weeks in air at room temperature. Brown spots were confirmed in the hydrophilic ointment containing 5% simple hydroquinone (far left), but no such coloration was found in the other ointments. Thus, formation of crystalline molecular complexes of hydroquinone with these surfactants clearly prevented oxidation of and stabilized hydroquinone.

### Example 5

### Visual Observation Test for Skin Reaction

A simple surfactant and surfactant/hydroquinone molecular complex crystals were kneaded with white vaseline, and used for a patch test with a patch tester (Torii Yakuhin) on the back of a 30-year-old woman. Skin reactions such as eruption, redness, edema or papules were visually noted 48 hours after applying each patch. Skin reactions were also confirmed after 72 hours.

The samples used for the patch test were as follows (where the percentage values indicate the proportions of the surfactant or molecular complex crystals).

1. 8% simple DM

2. 2% simple DM

3. 0.2% simple DM

4. 0.02% simple DM

5. 4% DM/HQ molecular complex crystals

6. 0.3% DM/HQ molecular complex crystals

7. 8% simple CDBAC

8. 2% simple CDBAC

9. 0.2% simple CDBAC

10. 0.02% simple CDBAC

11. 10% CDBAC/HQ molecular complex crystals

12. 4% CDBAC/HQ molecular complex crystals

13. 0.3% CDBAC/HQ molecular complex crystals

14. 0.05% CDBAC/HQ molecular complex crystals

15. Control

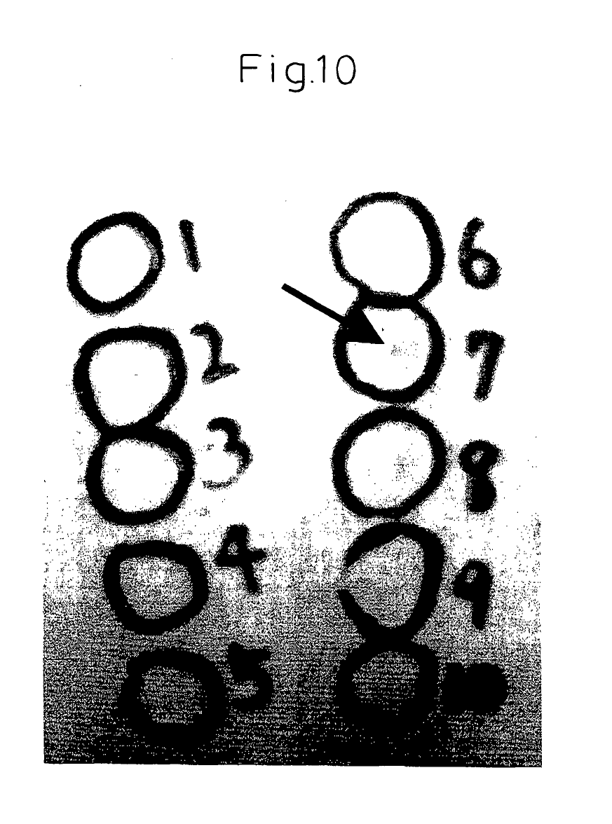

Results: FIGS. 10 and 11 shows the results after 48 hours from the start of the patch test. Although some redness was found at the center area of No. 7, virtually no change was observed and the overall evaluation was negative.

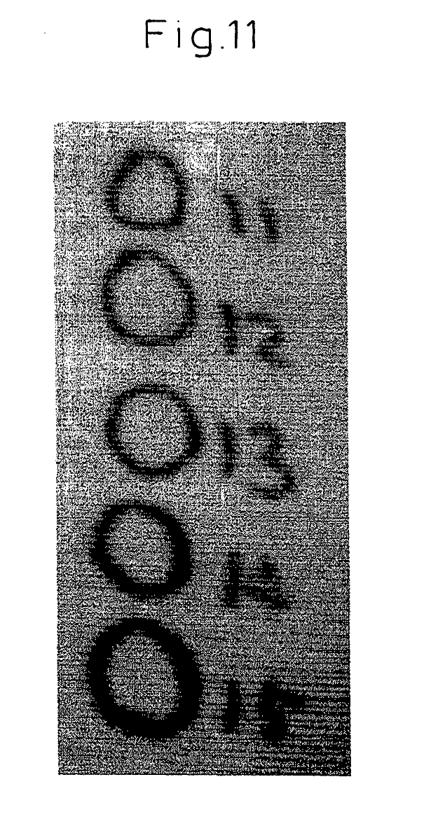

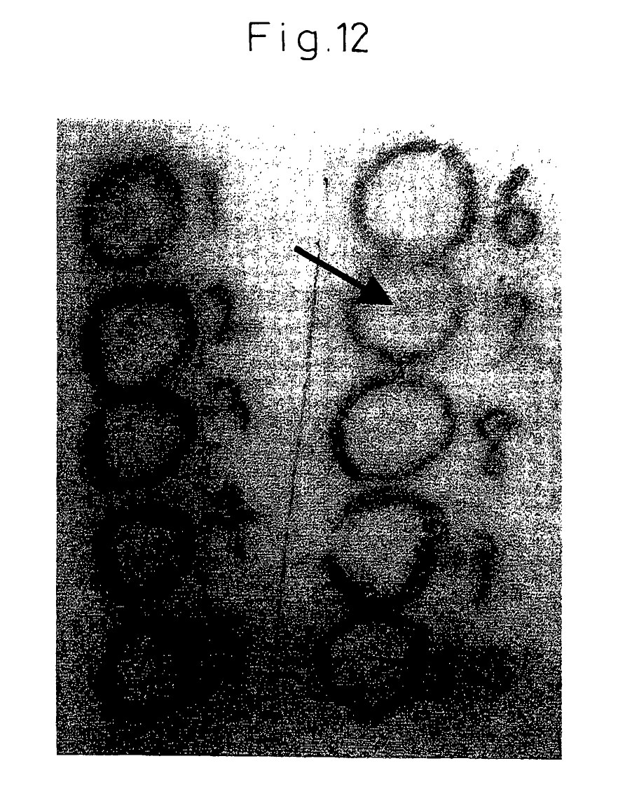

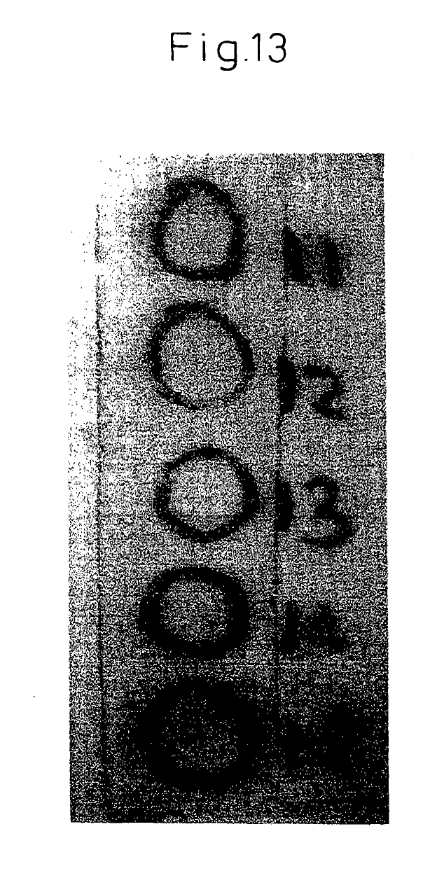

FIGS. 12 and 13 show the results after 72 hours from the start of the patch test. Some redness remained in the center area of No. 7, but the overall evaluation was negative.

Since no skin reaction was observed in any of the molecular complex crystals (Nos. 5-6, 10-14), it was concluded that the hydroquinone/surfactant molecular complex crystals of the invention have low skin irritability.

### Example 6

### Formulation of 50 g of Cream Containing 2% Hydroquinone

This example demonstrates a method of producing an external application skin cream requiring addition of water (aqueous phase).

Constituent components (50 g cream):

| | | | |
|---|---|---|---|
| 1. | Mineral oil | 8.0 | g |
| 2. | White vaseline | 3.0 | g |
| 3. | Liquid paraffin | 3.0 | g |
| 4. | Polyoxyethylene (2) stearyl ether | 1.5 | g |
| 5. | Polyoxyethylene stearyl ether stearate | 1.5 | g |
| 6. | Jojoba oil | 2.0 | g |
| 7. | Glycol distearate | 1.0 | g |
| 8. | Polyoxyethylene (25) stearyl ether | 0.5 | g |
| 9. | Polyoxyethylene isostearyl ether | 0.5 | g |
| 10. | Sorbitan tristearate | 0.5 | g |
| 11. | Octamethylcyclotetrasiloxane | 1.0 | g |
| 12. | Tristearin | 1.0 | g |
| 13. | Stearic acid | 1.5 | g |
| 14. | Squalane | 4.6 | g |
| 15. | Cetanol | 1.5 | g |
| 16. | Complex | 3.4 | g |
| 17. | Glycerin | 0.39 | g |
| 18. | 1,3-Butanediol | 3.5 | g |
| 19. | Trehalose | 0.1 | g |
| 20. | Citric acid | 1.5 | g |
| 21. | EDTA-2Na | 0.01 | g |
| 22. | Purified water | 10.0 | g |

Procedure:
(1) Preparation of First Oil Phase (A)

a. The hydroquinone and surfactant molecular complex (hereinafter referred to as “complex”) is micronized (≦80 mech).

b. The complex (16) is added to about 6 g of mineral oil (1) and mixed therewith at 75-80° C.

c. White vaseline (2) is added and after homogenization, components (4) and (5) are added prior to kneading for 10-15 minutes.

d. Liquid paraffin (3) is added and the entire mixture is homogenized.

e. The prepared phase is returned to room temperature.

(2) Preparation of second oil phase (B)

The remaining mineral oil (1) is combined with components (6)-(15) at 70-75° C. to prepare an oil phase.

The silicon-based surfactant (11) is added at this point.

(3) Preparation of aqueous phase (C)

Components (17)-(22) are mixed at 60-70° C. to obtain a homogeneous aqueous solution.

(4) Preparation of emulsion base (D)

The aqueous phase (C) is slowly added in small portions at a time to the second oil phase (B) while stirring for approximately 10 minutes. The addition is carried out at 75-80° C., and upon completion of the addition the mixture is gradually returned to room temperature while stirring.

(5) Preparation of cream (E)

Once the emulsion base (D) has reached room temperature, the first oil phase (A) is added in small portions at a time without heating, while mixing for approximately 10 minutes.

### Example 7

### Room Temperature Storage Stability of Hydroquinone/Surfactant Molecular Complex-Containing Cream Produced According to Example 6

According to the conventional method, the main component is dispersed in an oil phase, or in other words, the aforementioned first oil phase (A) and second oil phase (B) are prepared together and combined with the aqueous phase (C) to produce a cream. Separately, in the same manner as Example 6, an oil phase obtained by uniformly dispersing the main component (first oil phase (A)) beforehand is added to an emulsion base (D) also prepared beforehand, to produce a cream. A storage stability test was conducted in an air thermostatic chamber to confirm the effect of the method of Example 6 compared to the conventional method, giving the following results.

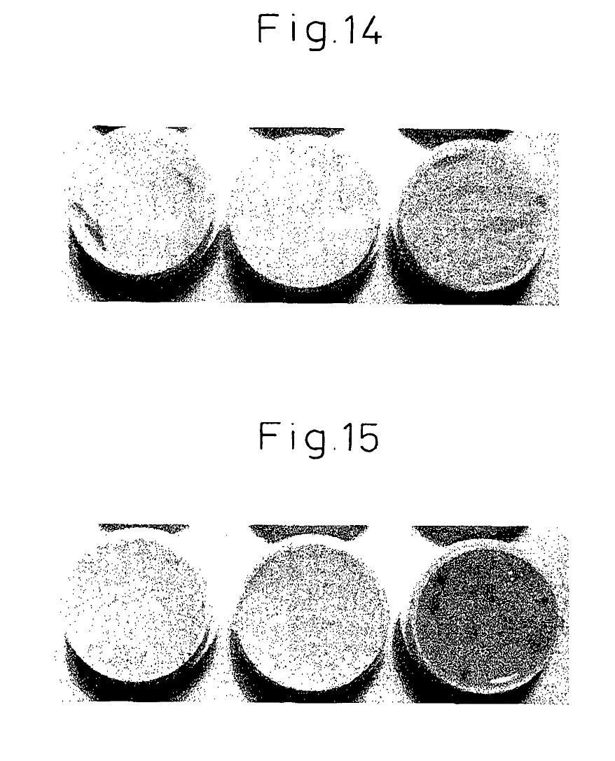

FIG. 14 shows the outer appearances of the creams at the time of preparation. The leftmost cream is the complex-containing cream prepared by the conventional method, the center cream is the complex-containing cream produced by the method of Example 6, and the rightmost cream is a cream containing simple hydroquinone, prepared according to the conventional method. No coloration or brown spots were present in any of the creams at the time of preparation.

FIG. 15 shows the outer appearances of the creams after standing at 40° C. for 24 hours. The arrangement of the leftmost, center and rightmost creams is the same as in FIG. 14. Brown spots were observed in the rightmost cream (simple hydroquinone, conventional method).

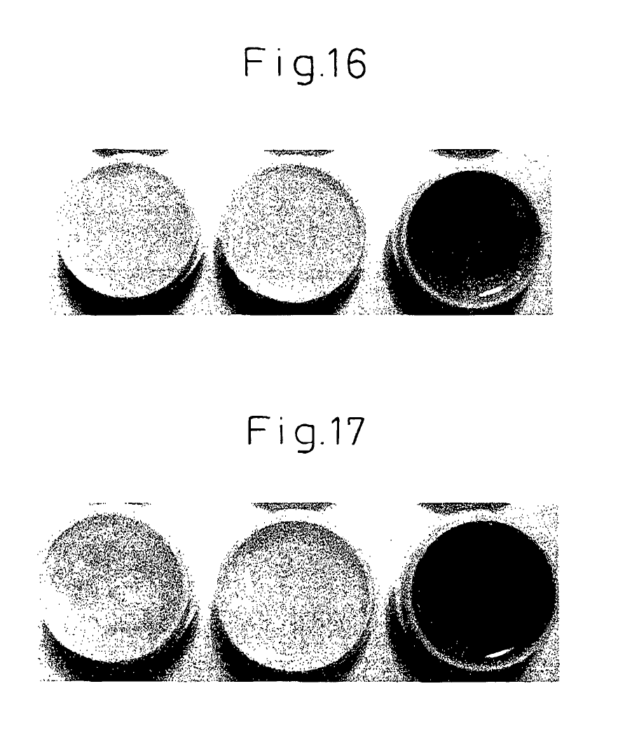

FIG. 16 shows the outer appearances of the creams after standing at 40° C. for 72 hours. The arrangement of the leftmost, center and rightmost creams is the same as in FIG. 14. Brown spots were observed in the leftmost cream (complex, conventional method), while significant brown coloration was observed in the rightmost cream (simple hydroquinone, conventional method). Cream separation was also observed in both the leftmost and rightmost creams.

FIG. 17 shows the outer appearances of the creams after standing at 40° C. for 110 hours. The arrangement of the leftmost, center and rightmost creams is the same as in FIG. 14. Brown spots and coloration were observed in the leftmost cream (complex, conventional method), while significant brown coloration was observed in the rightmost cream (simple hydroquinone, conventional method). The brown spots and coloration had clearly further progressed compared to the same after standing for 72 hours shown in FIG. 16. On the other hand, virtually no such coloration was seen in the center cream (complex, Example 6), and therefore the hydroquinone/surfactant molecular complex in the cream produced by the method of Example 6 clearly had maintained long-term stability in its crystal structure.

### Example 8

### Measurement of Coloration in Cream of Example 7 using Differential Colorimeter

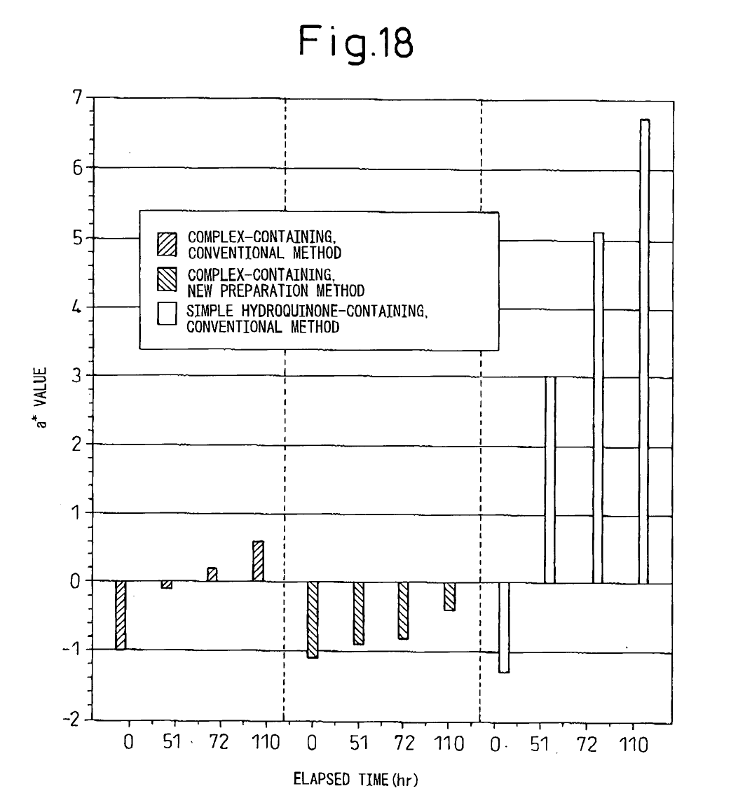

As shown in FIG. 18, using the conventional method (complex-containing or simple hydroquinone-containing) produced redness (a* value) in the cream toward the + end. However, no change in the a* value toward the + end was seen with the method of Example 6 (new preparation method).

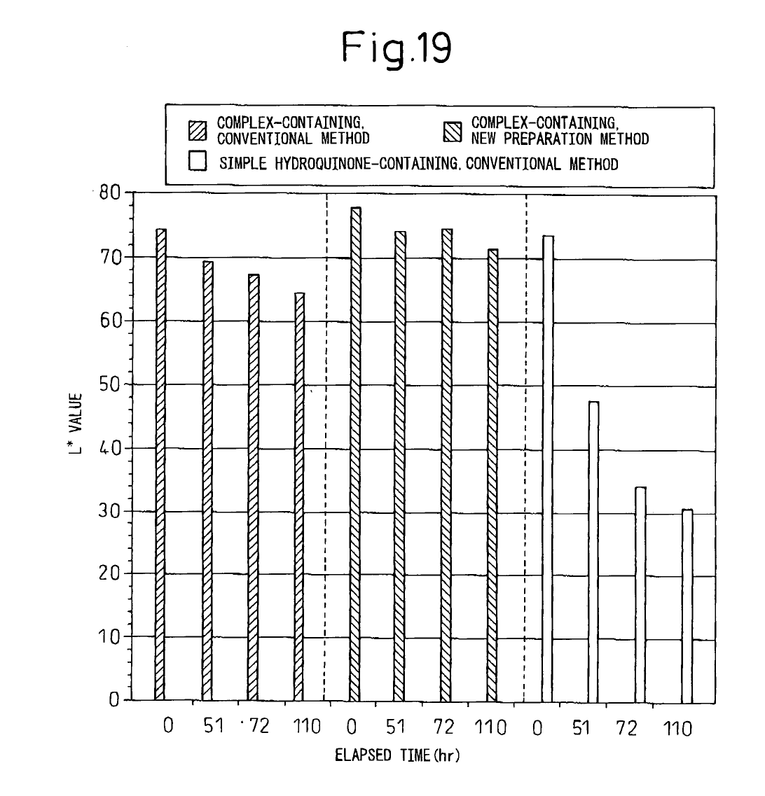

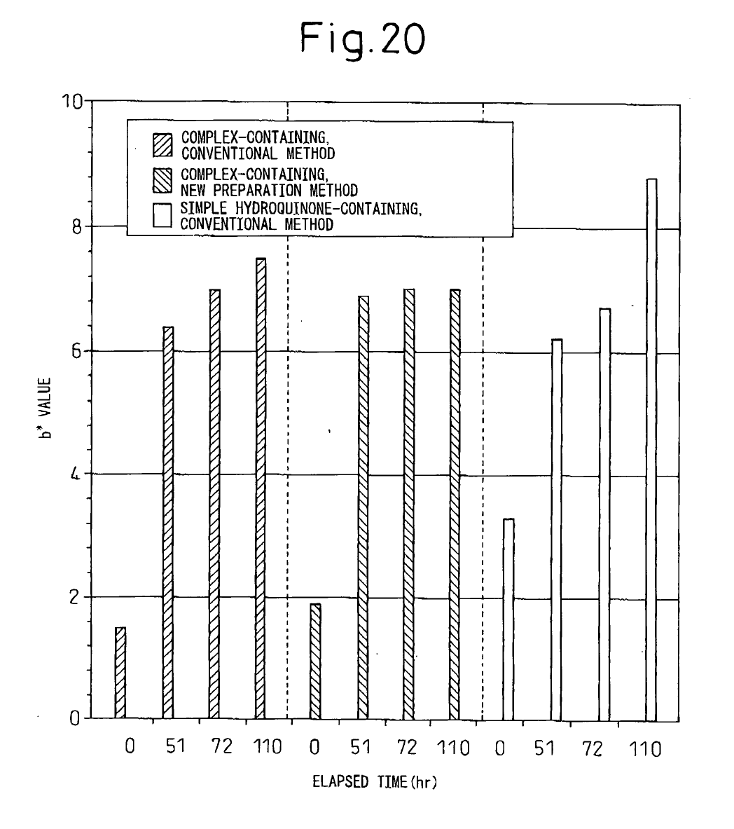

As shown in FIG. 19, using the conventional method (complex-containing or simple hydroquinone-containing) resulted in reduced whiteness of the cream (lightness: L* value), whereas the new preparation method did not result in a reduction in the L* value.

### Example 9

### Cream Light Resistance Test Under Deaeration

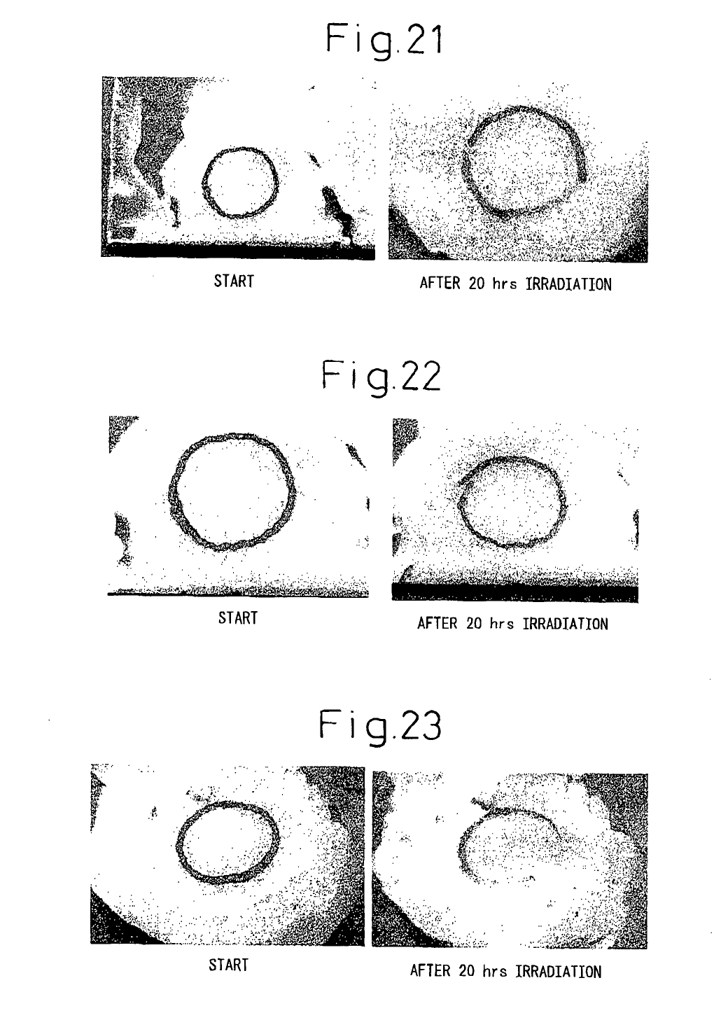

A light resistance test was carried out for the complex-containing cream prepared by the (disclosed) preparation method of Example 6, the complex-containing cream prepared by the conventional preparation method (conventional emulsification method) and the simple hydroquinone cream prepared by the conventional preparation method (conventional emulsification method) (see FIGS. 21, 22 and 23, respectively). Each cream was placed in a polyethylene bag and the bag was sealed using a Vacuum Sealer after sufficient deairing. It was then irradiated for 20 hours with a xenon lamp (30 mW/cm² dose), and the deterioration of the cream under light was visually observed based on coloration and measured using a differential calorimeter.

Increased redness was visually confirmed for the simple hydroquinone cream obtained by the conventional method (see FIG. 23).

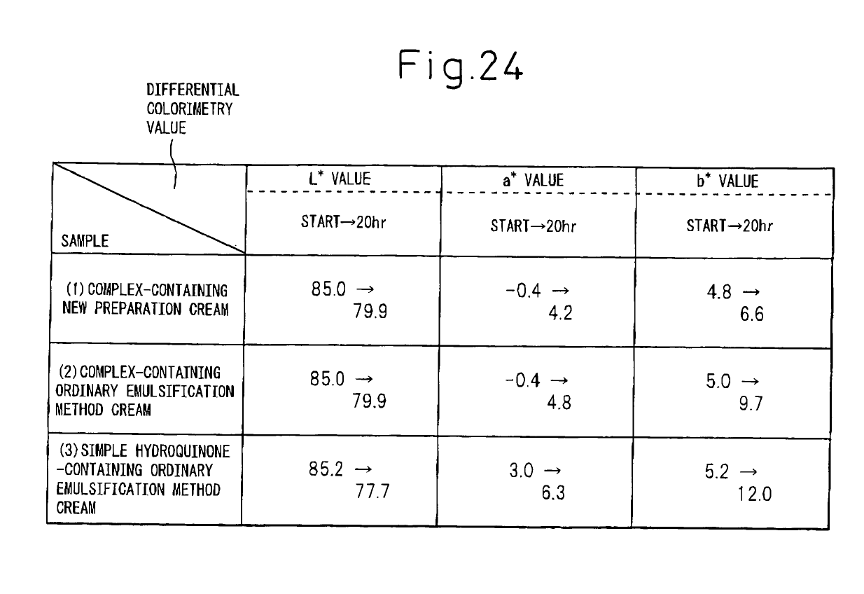

FIG. 24 shows the results of measuring coloration using a differential calorimeter. The simple hydroquinone cream obtained by the conventional method clearly had a larger increase in a* value and b* value compared to the other creams (see last row (3)). Also, the disclosed preparation method (1) clearly resulted in a smaller increase in a* value and b* value compared to the conventional method (2).

1. A whitening agent comprising a crystalline molecular complex composed of hydroquinone or a derivative thereof and a surfactant, characterized in that formation of said molecular complex improves the storage stability of the hydroquinone-containing whitening agent against heat, oxygen and light, while said hydroquinone is gradually released for a sustained whitening effect of said whitening agent.

2. A whitening agent according to claim 1, wherein said hydroquinone or its derivative is selected from the group consisting of hydroquinone, hydroquinone monobenzyl ether, hydroquinone monomethyl ether and hydroquinone monoethyl ether.

3. A whitening agent according to claim 1, wherein said hydroquinone or its derivative is hydroquinone.

4. A whitening agent according to claim 1, wherein said surfactant is selected from the group consisting of octadecyltrimethylammonium bromide (STAB), octadecyltrimethylarrmonium chloride (STAC), hexadecyltrimethylammonium bromide (CTAB ), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC), tetradecyldimethylbenzylammonium bromide (BZB), tetradecyldimethylbenzylammonium chloride (BZCL), dodecyltrimethylammonium bromide (LTAB), didecyltrimethylamL-,ioniumchloride (LTAC), decyltrimethylammonium bromide (DTAB), decyltrimethylmmonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

5. A whitening agent according to claim 1, wherein said surfactant is selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

6. A whitening agent according to claim 1, wherein said surfactant is CDBAC.

7. The use of a crystalline molecular complex composed of hydroquinone or a derivative thereof and a surfactant for production of a whitening agent, characterized in that formation of said molecular complex improves the storage stability of the hydroquinonecontaining whitening agent against heat, oxygen and light, while said hydroquinone is gradually released for a sustained whitening effect of said whitening agent.

8. The use according to claim 7, wherein said hydroquinone or its derivative is selected from the group consisting of hydroquinone, hydroquinone monobenzyl ether, hydroquinone monomethyl ether and hydroquinone monoethyl ether.

9. The use according to claim 7, wherein said hydroquinone or its derivative is hydroquinone.

10. The use according to claim 7, wherein said surfactant is selected from the group consisting of octadecyltrimethylammonium bromide (STAB), octadecyltrimethylammonium chloride (STAC), hexadecyltrimethylammonium bromide (CTAB), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC), tetradecyldimethylbenzylammonium bromide (BZB), tetradecyldimethylbenzylammonium chloride (BZCL), dodecyltrimethylammonium bromide (LTAB), dodecyltrimethylammonium chloride (LTAC), decyltrimethylammonium bromide (DTAB), decyltrimethylammonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

11. The use according to claim 7, wherein said surfactant is selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

12. The use according to claim 7, wherein said surfactant is CDBAC.

13. A whitening method for skin wherein a whitening agent comprising a crystalline molecular complex composed of hydroquinone or a derivative thereof and a surfactant is applied to pigmented skin, the method being characterized in that formation of said molecular complex improves the storage stability of the hydroquinonecontaining whitening agent against heat, oxygen and light, while said hydroquinone is gradually released for a sustained whitening effect of said whitening agent.

14. A whitening method according to claim 13, wherein said hydroquinone or its derivative is selected from the group consisting of hydroquinone, hydroquinone monobenzyl ether, hydroquinone monomethyl ether and hydroquinone monoethyl ether.

15. A whitening method according to claim 13, wherein said hydroquinone or its derivative is hydroquinone.

16. A whitening method according to claim 13, wherein said surfactant is selected from the group consisting of octadecyltrimethylammonium bromide (STAB), octadecyltrimethylammonium chloride (STAC), hexadecyltrimethylammonium bromide (CTAB), hexadecyltrimethylammonium chloride (CTAC), tetradecyltrimethylammonium bromide (MTAB), tetradecyltrimethylammonium chloride (MTAC), hexadecyldimethylbenzylammonium bromide (CDBAB), hexadecyldimethylbenzylammonium chloride (CDBAC), tetradecyldimethylbenzylammonium bromide (BZB), tetradecyldimethylbenzylammonium chloride (BZCL), dodecyltrimethylammonium bromide (LTAB), dodecyltrimethylammonium chloride (LTAC), decyltrimethylammonium bromide (DTAB), decyltrimethylammonium chloride (DTAC), dodecyldimethylbenzylammonium bromide (LDBAB), dodecyldimethylbenzylammonium chloride (LDBAC), decyldimethylbenzylammonium bromide (DDBAB), decyldimethylbenzylammonium chloride (DDBAC) and n-dodecyl-β-D-maltoside (DM).

17. A whitening method according to claim 13, wherein said surfactant is selected from the group consisting of octadecyltrimethylammonium chloride (STAC), hexadecyltrimethylammonium chloride (CTAC), tetradecyltrimethylammonium chloride (MTAC), hexadecyldimethylbenzylammonium chloride (CDBAC) and tetradecyldimethylbenzylammonium chloride (BZCL).

18. A whitening method according to claim 13, wherein said surfactant is CDBAC.

19. A process for production of a whitening agent according to claim 1, which process comprises the following steps:
dispersing a crystalline molecular complex composed of hydroquinone or a derivative thereof and a surfactant in a first oil phase;
preparing a second oil phase;
preparing an aqueous phase;
adding said aqueous phase to the second oil phase and stirring to form an emulsion; and
adding said first oil phase containing said molecular complex to said emulsion and stirring to obtain a whitening cream containing the said molecular complex.

20. The process of claim 19, wherein said first oil phase contains mineral oil, white vaseline, liquid paraffin, polyoxyethylene (2) stearyl ether and/or polyoxyethylene stearyl ether stearate.

21. The process of claim 19, wherein said second oil phase contains mineral oil, jojoba oil, glycol distearate, polyoxyethylene (25) stearyl ether, polyoxyethylene isostearyl ether, sorbitan tristearate, octamethylcyclotetrasiloxane, tristearin, stearic acid, squalane and/or cetanol.

22. The process of claim 19, wherein said aqueous phase contains glycerin, 1,3-butanediol, trehalose, citric acid and/or EDTA-2Na, and purified water.
